(19) United States
(12) Patent Application Publication
(10) Pub. No.: US 2024/0164888 A1
(43) Pub. Date: May 23, 2024

(54) SYSTEMS, METHODS, AND DEVICES FOR TREATING A DISEASED OR OTHERWISE DAMAGED TRICUSPID VALVE

(71) Applicant: Innoventric Ltd., Rehovot (IL)

(72) Inventors: Amir DANINO, Rehovot (IL); Yair PICHERSKY, Tel Aviv (IL); Eyal KISLEV, Tel Aviv (IL)

(21) Appl. No.: 18/277,215

(22) PCT Filed: Feb. 15, 2022
(86) PCT No.: PCT/IB2022/051347

§ 371 Date: Aug. 14, 2023

Related U.S. Application Data
(60) Provisional application No. 63/149,555, filed on Feb. 15, 2021.

(51) Int. Cl. A61F 2/07; A61F 2/06; A61F 2/24; A61F 2/95; A61F 2/966; A61N 1/362

(52) U.S. Cl. CPC A61F 2/07; A61F 2/2418; A61F 2/966; A61F 2002/061; A61F 2002/9528; A61F 2002/9534; A61N 1/362

(57) ABSTRACT

Embodiments of the subject disclosure are directed a tricuspid valve treatment device, as well as associated methods for treating and/or replacing a diseased or otherwise damaged tricuspid valve using, for example, such disclosed devices and systems) is provided. In some embodiments, the device includes a lateral outflow port configured for arrangement after implantation in the right atrium, or right atrium and right ventricle, and configured for the fixation of a surgical or transcatheter valve, where such valve can be placed after the tricuspid valve treatment device is implanted.

### CROSS-REFERENCE TO RELATED APPLICATIONS

This application claims the benefit of U.S. Provisional Patent Application No. 63/149,555, filed on Feb. 15, 2021, the content of which is herein incorporated by reference in its entirety.

The present disclosure is also related to PCT Application No. PCT/IB2017/050534, filed Feb. 1, 2017, and PCT Application No. PCT/IL2019/050658, filed Jun. 7, 2019. Each of these disclosures is herein incorporated by reference in its entirety.

### FIELD OF THE DISCLOSURE

The present disclosure generally relates to medical systems, apparatuses, devices, and methods for implantation in the heart thereof, and particularly, but not only, to a stent-based device for treating diseases and/or malfunctions of the tricuspid valve.

### BACKGROUND

The tricuspid valve prevents back flow of blood from the right ventricle into the right atrium when it closes during ventricular systole and allows blood to flow from the right atrium into the right ventricle when it opens during ventricular diastole.

An insufficient tricuspid valve causing tricuspid regurgitation may occur from tricuspid annular dilation and right ventricular enlargement. Tricuspid regurgitation is often secondary to heart failure from myocardial or valvular causes, right ventricular volume or pressure overload and dilation of cardiac chambers. Tricuspid regurgitation causes right atrial overload that is transmitted to the superior and inferior vena cava and their tributaries. Eventually, this leads to hepatic congestion, ascites, anasarca, peripheral edema, and other clinical symptoms of congestive heart failure. If untreated, significant tricuspid regurgitation frequently leads to heart failure and death.

Clinically available treatments for tricuspid regurgitation include open heart surgery and/or medication. However, open heart surgery for the replacement and/or repair of the tricuspid valve is rarely carried out, mainly due to its high mortality and morbidity rates. Medication, on alternatively, may not solve the problem and may allow the disease to progress, leaving patients with a deteriorated quality of life and cardiac function.

Due to the high surgical risk of tricuspid valve replacement and/or repair, currently, most tricuspid regurgitation patients are deemed inoperable. This results in an extremely large number of untreated patients with significant tricuspid regurgitation.

### BRIEF SUMMARY

According to an embodiment, the present disclosure relates to a stent graft device configured for implantation into the inferior vena cava and the superior vena cava of a patient to treat a diseased or otherwise damaged tricuspid valve of the patient, the device comprising a tubular body having a longitudinal axis and configured for implantation within the superior vena cava and the inferior vena cava, such that, upon implantation, a first end and associated first end portion of the tubular body is arranged in the inferior vena cava, and a second end and associated second end portion of the tubular body is arranged in the superior vena cava, a first outflow port configured for arrangement after implantation in the right atrium and right ventricle, and configured for the fixation of a surgical or transcatheter valve, wherein the first outflow port is arranged laterally to the longitudinal axis, and optionally, automatically deploys upon exiting a delivery catheter.

In an embodiment, the first outflow port is arranged laterally to the longitudinal axis and at an oblique angle to the longitudinal axis, the first outflow port is arranged lateral to the longitudinal axis and at an oblique angle to the longitudinal axis, with a distal end of the first outflow port pointing in a direction toward the base of the heart, and/or the first outflow port is arranged lateral to the longitudinal axis and at an oblique angle to the longitudinal axis, with a distal end of the first outflow port pointing in a direction toward the apex of the heart.

In an embodiment, the device further comprises a material configured to at least partially cover the tubular body, wherein the material is arranged on the tubular body such that it prevents blood flow through at least side portions of the tubular body, and wherein arrangement of the material on the tubular body is configured to allow inflow of blood from the azygos/innominate and/or hepatic veins.

In an embodiment, the device further comprises a pacemaker/ICD lead port and/or valve (PLP) configured to receive for pacemaker/ICD lead passage, wherein the PLP is arranged on a side of the tubular body, wherein the first outflow port includes at least one of a first restriction and a second restriction, each configured to prevent migration of a valve arranged therein, wherein the first outflow port includes at least one restriction, configured as a bottleneck structure, the bottleneck structure being within the first outflow port, or, the first outflow port is configured with an hourglass shape such that the bottleneck structure is within the hourglass structure, the bottleneck structure configured to prevent migration of a valve arranged within the first outflow port, wherein the first outflow port includes a first stopper position, arranged on a distal end of the first outflow port, the first stopper portion configured to prevent migration of a valve arranged within the first outflow port away from the tubular body, wherein the first outflow port includes a first stopper portion, arranged on a proximal end of the first outflow port, the first stopper portion configured to prevent migration of a valve arranged within the first outflow port toward the tubular body, wherein the first outflow port includes a first stopper position arranged on a distal end of the first outflow port, the first stopper portion configured to prevent migration of a valve arranged within the first outflow port away from the tubular body, and a second stopper portion, arranged on a proximal end of the first outflow port, the second stopper portion configured to prevent migration of a valve arranged within the first outflow port toward the tubular body, wherein the first outflow port includes a limiting structure arranged on a distal end of the first outflow port, the limiting structure configured to prevent migration of a valve arranged within the first outflow port away from the tubular body, wherein at least one of a proximal end and a distal end of the first outflow port includes a diameter less than a diameter of a central portion of the first outflow port, which are configured to prevent migration of a valve arranged within the first outflow port, and/or wherein the first outflow port includes a stent structure configured as a spring and configured to provide an inward radial force on the first outflow port.

In an embodiment, the device further comprises a skirt comprised of sealing material and arranged at least one of in and around the tubular body, wherein the skirt is configured to prevent a backflow of blood from the right atrium to the inferior vena cava, wherein the skirt includes a flange shape having an inner diameter which is attached to the tubular body proximate to the first end, and outer diameter for interfacing with the inferior vena cava, the other diameter spaced longitudinally away from the inner diameter in a direction toward the second end, outer diameter including a straight edge, wherein the skirt includes an outer diameter which is for interfacing with the inferior vena cava, and an inner diameter spaced longitudinally away from the outer diameter in a direction toward the second end, the outer diameter includes a curved edge, wherein the skirt includes a donut shape including an outer diameter configured to interface with the inferior vena cava, and an inner diameter radially spaced away from the outer diameter and configured for attachment to the tubular body, wherein the skirt includes a flange shape, which includes an outer diameter arranged proximate the first end and for interfacing with the inferior vena cava, and an inner diameter which is attached to the tubular body and spaced longitudinally away from the inner diameter in a direction toward the second end, the outer diameter including a straight edge, wherein the first port is self-expanding and/or includes a predetermined shape, wherein the device is configured for delivery to the inferior vena cava and/or superior vena cava via a sheathing delivery device, and wherein, the device is optionally configured for re-sheathing by the sheathing delivery device for at least one of repositioning, redeploying, and removal, wherein at least the first outflow port is configured to be re-sheathed in a sheathing device, wherein the first outflow port includes a stent structure configured to support a valve device placed within, on, or adjacent to the first outflow port, wherein the first outflow port is configured with a length and/or shape such that upon implantation, the first port reaches the right atrium, wherein the first outflow port includes at least one fenestration along a first contour (right atrial contour) or by having a shape that allows flow from the sides of a valve within, on, or adjacent the first port, wherein the first outflow port is designed to prevent retrograde jet flows from reaching a/the valve placed within, on, or adjacent to the first outflow port, wherein, upon implantation of the device, at least a portion of the first port is arranged within the RA, and optionally, at least a portion of the first outflow port protrudes into the right ventricle via the tricuspid valve, wherein the tubular body includes a stent comprising a scaffold structure configured to operate in a first compressed mode, such that the device fits within a delivery catheter, and a second expanded mode, such that, upon placement of the device at an implantation site, the device is configured to self-expand therein, or expand via a balloon, and/or wherein the tubular body includes a rigidity that varies at different portions thereof.

According to an embodiment, the present disclosure relates to a method of delivering the device disclosed herein via at least one of a transcatheter delivery device and a transapical delivery device.

According to an embodiment, the present disclosure relates to a method of implanting a valve within a tubular stent, the method comprising delivering a stent graft device to the heart of a patient, such that upon implantation, a first end and associated first end portion of the tubular body is arranged in the IVC, a second end and associated second end portion of the tubular body is arranged in the SVC, and the first outflow port is arranged at least in the right atrium, delivering a stented valve up through one or the other of the IVC and SVC so as to be arranged within at least a portion of the first outflow port, and balloon expanding the stented valve within the first outflow port. In an embodiment, the expanding the stented valve within the first outflow port includes greater expansion on at least one first portion of the stented valve than remaining portions of the valve, wherein the first portion comprises at least one of and optionally two of a proximal portion, a center portion, and a distal portion of the stented valve.

According to an embodiment, the present disclosure relates to a system, device, apparatus, or method according to any of the embodiments disclosed herein.

### BRIEF DESCRIPTION OF THE SEVERAL VIEWS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structural similar elements).

FIG. 1A is a side view of an implanted tricuspid treatment device into the inferior vena cava, the superior vena cava, and the right atrium, according to embodiments.

FIG. 1B is a side view of a tricuspid treatment device, according to embodiments.

FIG. 2 is a side/perspective view of a tricuspid treatment device, according to embodiments.

FIG. 3 is a side view of an implanted tricuspid treatment device into the inferior and superior vena cava and right atrium, and also illustrating sealing structure, according to embodiments.

FIG. 4A and FIG. 4B illustrate two configurations of skirt sealing structures, according to embodiments.

FIG. 5A and FIG. 5B illustrate two configurations of skirt sealing structures, according to embodiments.

FIG. 6A through FIG. 6D are side views of ports or valves configured for placement of other devices, through a wall of the tricuspid treatment device, according to embodiments.

FIG. 7A and FIG. 7B are side views of a tricuspid treatment device, which includes openings in an outflow port thereof, according to embodiments.

FIG. 8 is a cross-sectional side view of a tricuspid treatment device, and a stopper-device arranged adjacent to an outflow port thereof, according to embodiments.

FIG. 9 is a side view of an implanted tricuspid treatment device into the inferior vena cava, the superior vena cava, the right atrium, and the right ventricle, according to embodiments.

FIG. 10A and FIG. 10B illustrate side views of a tricuspid treatment device showing different configurations of the outflow port and components thereof, according to embodiments.

FIG. 11A through FIG. 11D illustrate side views of a tricuspid treatment device showing different configurations of the outflow port and components thereof, according to embodiments.

FIG. 11E through FIG. 11G are images during implantation of the implantable tricuspid treatment device, according to embodiments.

FIG. 12 is a side view of a tricuspid treatment device illustrating a component contained among the outflow port thereof, according to embodiments.

### DETAILED DESCRIPTION

### Definitions

The term “a” or “an” refers to one or more of that entity, i.e. can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

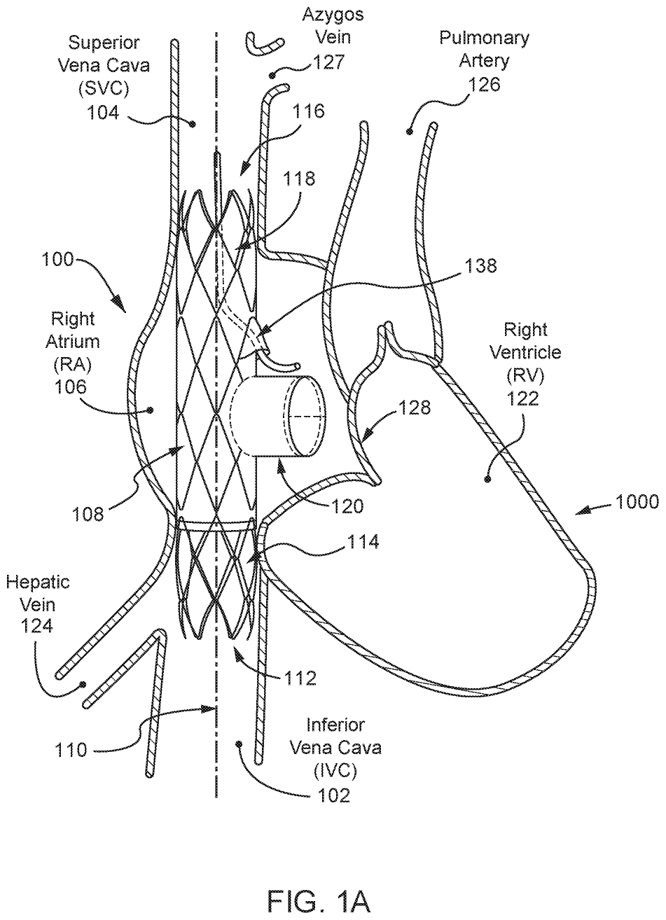

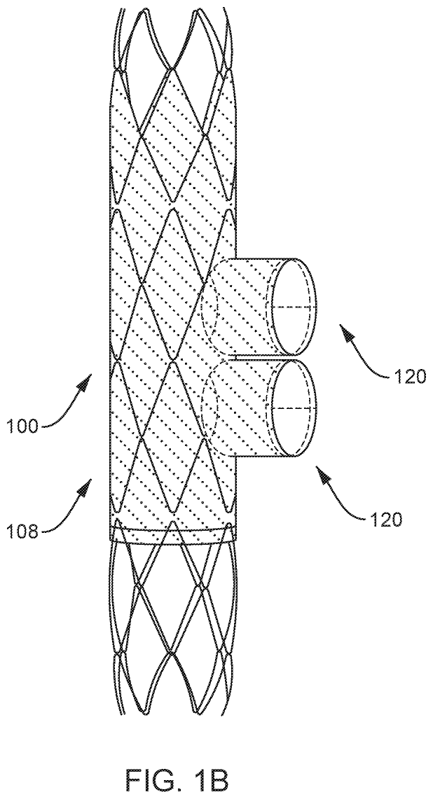

FIG. 1A is a side view of a heart, including the inferior vena cava 102, the superior vena cava 104, and the right atrium 106 of the heart 1000. The superior vena cava 104 returns blood from the upper half of the body and opens into the superior and posterior region of the right atrium 106 via a valve-less orifice which is directed inferiorly and anteriorly. The inferior vena cava 102, typically larger than the superior vena cava 104, returns blood from the lower half of the body, and opens into the inferior region of the right atrium 106, near the atrial septum. Its orifice is directed superiorly and posterior and protected by a valve of the inferior vena cava 102 (called the Eustachian valve).

The tricuspid valve 128 is located between the right atrium 106 and the right ventricle 122. The coronary sinus opens into the right atrium 106, between the orifice of the inferior vena cava 102 and the atrioventricular opening. It returns blood from the substance of the heart 1000 and is protected by a semicircular valve, the valve of the coronary sinus (also called the valve of Thebesius).

During ventricular systole, pressure is increased inside of the ventricles. During normal conditions in, for instance, the right side of the heart, pressure in the right ventricle 122 rises to a level above that of the right atria 106, thus closing the tricuspid valve 128. However, when the tricuspid valve 128 is unable to close completely during ventricular systole, which may be the case in patients with heart disease, tricuspid regurgitation occurs, and blood flow from the right ventricle 122 returns to the right atrium 106 with the potential to return deoxygenated blood to venous circulation via the superior vena cava 104 and/or the inferior vena cava 102.

Though other valves of the heart (e.g., mitral valve, aortic valve), may be readily replaced and/or repaired during open heart surgery, the tricuspid valve 128 is difficult to repair surgically. The tissue surrounding the tricuspid valve 128 is highly mobile, making any repair or replacement technically demanding, and rates of morbidity and mortality of the procedure, at large, warrant consideration of other approaches.

According to embodiments, the present disclosure describes an implantable tricuspid treatment device that resides within the right atrium 106, the inferior vena cava 102, and the superior vena cava 104, providing the benefits of a tricuspid valve 128 repair and/or replacement without the risk associated with surgery there. Moreover, the implantable tricuspid treatment device can be delivered via catheter, thus further minimizing the complications of the cardiac intervention.

With reference now to the Figures, and in view of the above description, FIG. 1A is a side view of an implanted tricuspid treatment device (TTD) 100 positioned within the inferior vena cava 102, the superior vena cava 104, and the right atrium 106 of the heart 1000, according to embodiments.

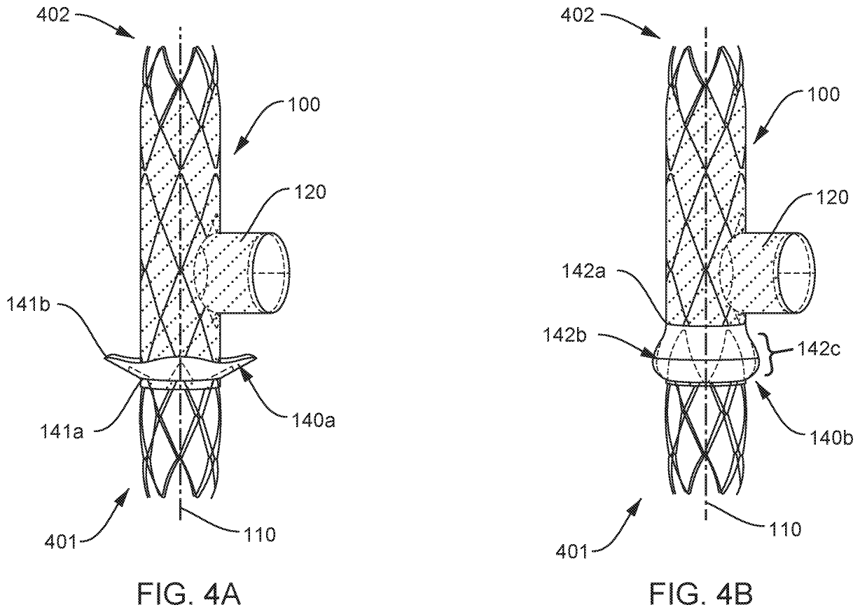

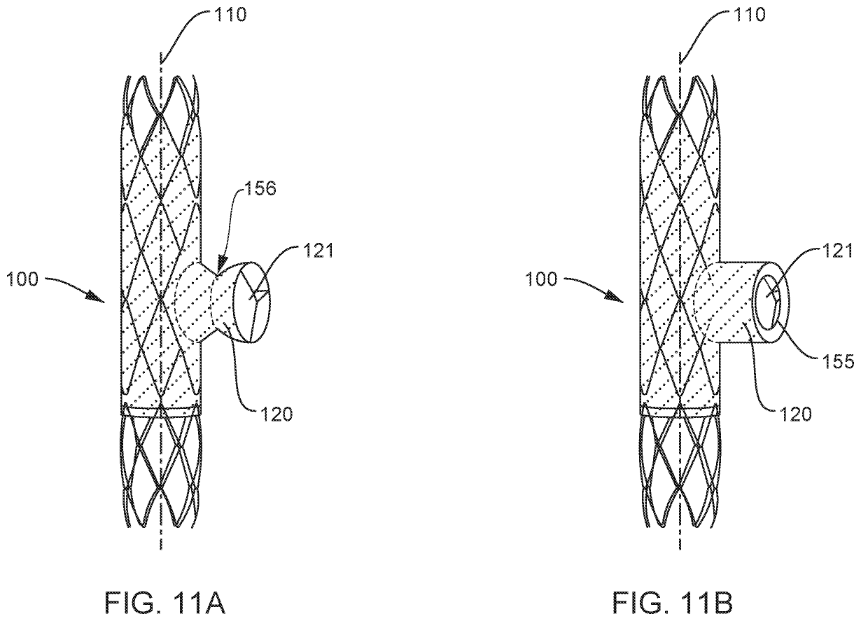

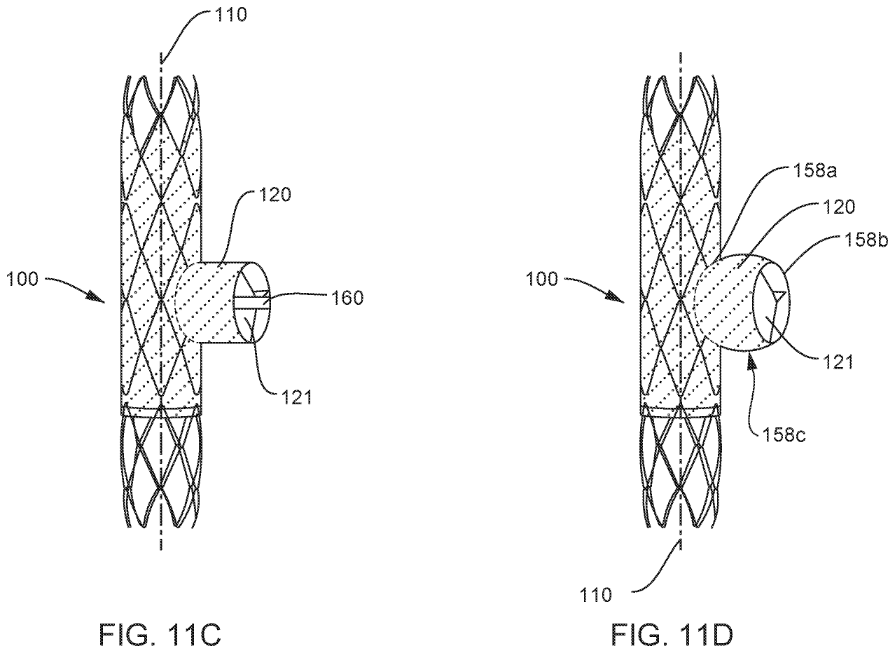

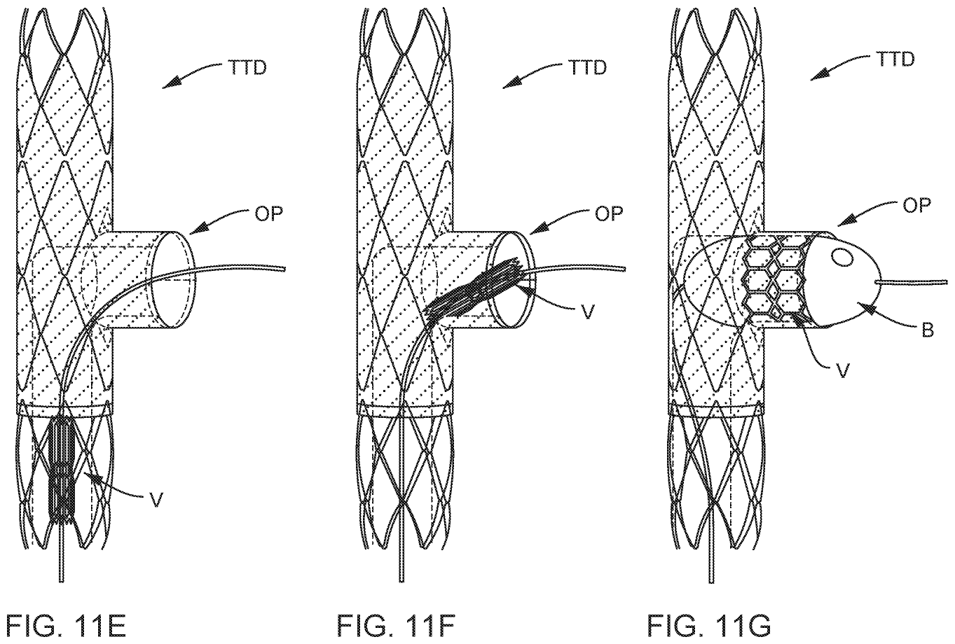

In some embodiments, the TTD 100 includes a tubular body 108, covered by a material (gray shading in the figures, see, e.g., FIGS. 4A-B, 5A-B, 6A-D, 7A-B, 8, 10A-B, 11A-11D, 12), and having a longitudinal axis 110. Upon implantation, a first end 112 and an associated first end portion 114 of the tubular body 108 are arranged in the inferior vena cava 102, and a second end 116 and an associated second end portion 118 of the tubular body 108 are arranged in the superior vena cava 104. The TTD 100 also includes at least one outflow port 120 (which can be referred to as a first outflow port) configured for arrangement after implantation in the right atrium 106, or in the right atrium 106 and the right ventricle 122. The first outflow port 120 of the TTD 100 may be configured for the fixation of a surgical or transcatheter valve (see, e.g., FIGS. 11A-11B, illustrating, cross-sectionally, a valve 121 arranged therein).

In some embodiments, the first outflow port 120 is one of a plurality of outflow ports arranged along the tubular body 108 of the TTD f100, as shown in FIG. 1B. In embodiments, one or more of the plurality of outflow ports may be arranged along the tubular body 108 and positioned within the right atrium 106. In embodiments, the number of outflow ports can be based on dimensions of each outflow port and in view of a magnitude of tricuspid regurgitation and pressure gradients within the heart 1000. For instance, if basal pressure in the right atrium 106 are significantly elevated due to tricuspid regurgitation from the right ventricle 122, the number of outflow ports and/or a size of each outflow port may need to be adjusted to accommodate, and function within, the elevated pressure environment.

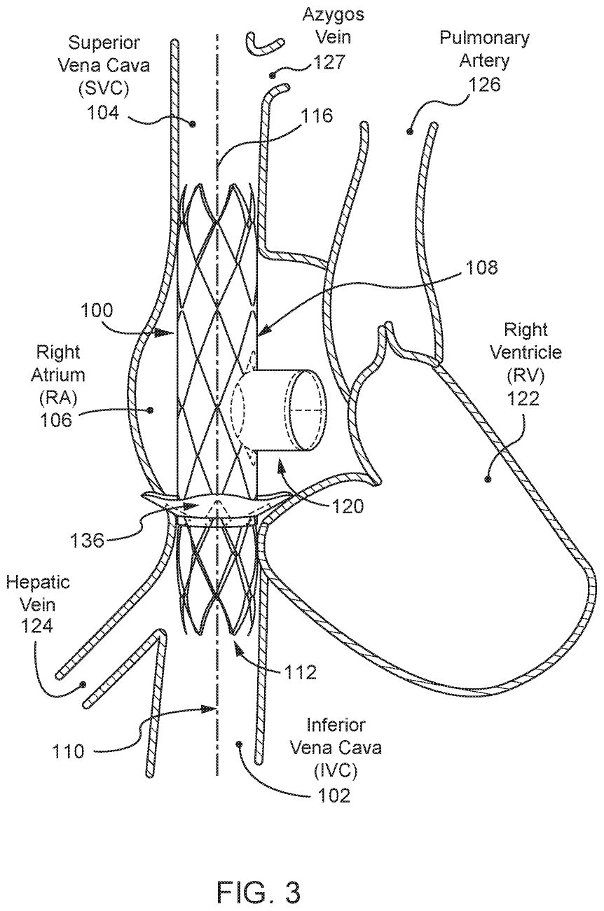

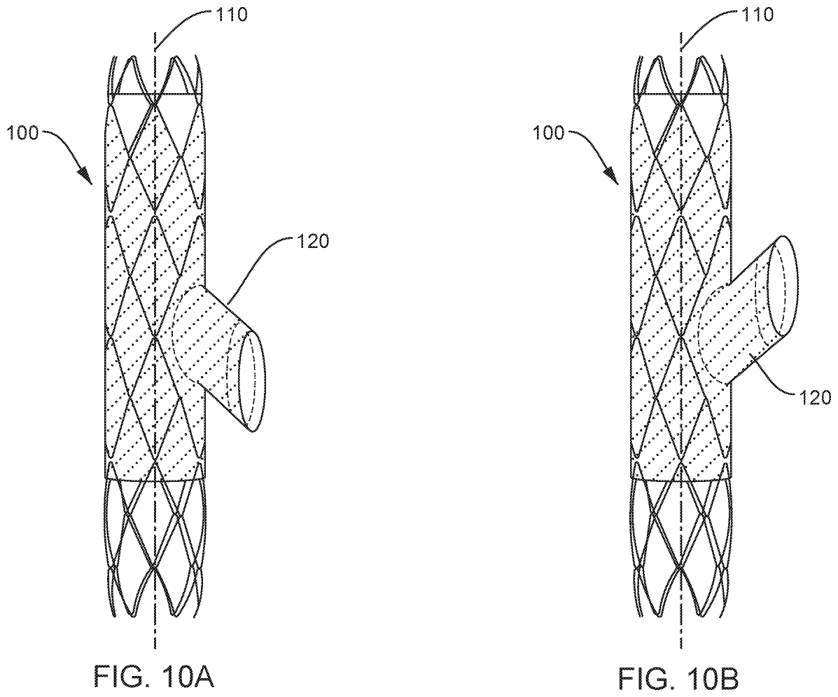

In some embodiments, the first outflow port 120 is arranged laterally to the longitudinal axis 110 (but may also be at an oblique angle, see, e.g., FIGS. 10A-B), and optionally, automatically deploys upon exiting a delivery catheter (not shown). FIG. 1A also illustrates the hepatic vein 124, the pulmonary artery 126, and the Azygos vein 127. The TTD 100 may also include a valve or port 138, for providing access to portions of the heart (for example), arranged on a side of the TTD 100, which may comprise, in some embodiments, a flap or cover, which can be biased in a closed position. Such access can be configured for, e.g., a pacing lead of a pacemaker device. FIG. 3 is another side view of an implanted TTD 100, like FIG. 1A, that illustrates the implanted TTD 100 positioned within the inferior vena cava 102, the superior vena cava 104, and the right atrium 106. FIG. 3 also illustrates a sealing structure, corresponding to a skirt element 136. In some embodiments, each of the superior and inferior ends of the TTD 100, and in particular the stent structure, may be curved inward (e.g., toward the longitudinal axis 110) so as to avoid damaging the superior vena cava 104 and the inferior vena cava 102, respectively (see curved portion of each end throughout all the figures).

It is worth noting that in some embodiments, the TTD 100 is configured as an anchoring device/mechanism for a surgeon to implant, and then which is used as a housing and/or anchor for a new prosthetic valve (e.g., to replace the function of a diseased or defective tricuspid valve) within a/the lateral outflow port 120 (of the device/tubular body 108), the prosthetic valve being implanted after the TTD 100 is implanted. In still other embodiments, the TTD 100 can be delivered with a prosthetic valve already in-place/affixed within the first outflow port 120. In fact, in embodiments including a prosthetic valve already in-place, the TTD 100 can be configured such that the in-place valve is configured to be removable/replaceable later. To this end, a new prosthetic valve can replace the already in-place valve. In other embodiments, a new prosthetic valve can be placed within the previously provided, already in-place prosthetic valve without removing the already in-place prosthetic valve. In embodiments, the above-described functionality can be had either with a TTD 100 being an anchoring device for later receival of a valve, or, a TTD 100 including an already in-place valve.

As one of skill in the art will appreciate, the stent structure of the TTD 100 can comprise a series of connected (integral or otherwise affixed to one another) struts which create a series of openings. Such a structure can be made of nitinol material. In some embodiments, the nitinol material enables the stent structure to at least radially compress to a fraction of its expanded diameter, so as to fit within a sheath of a delivery device and self-expand after removal therefrom. In some embodiments, such expansion can be aided by balloon expansion (or replace self-expansion).

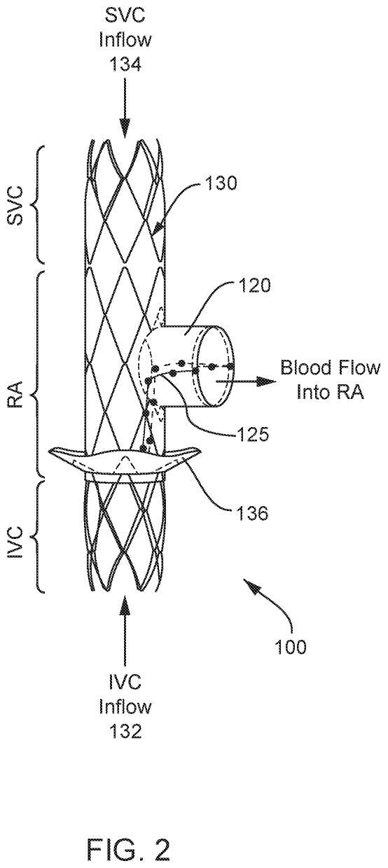

FIG. 2 is a side/perspective view of a TTD 100, according to some embodiments, which shows the stent structure 130, inflow 132 from the inferior vena cava, inflow 134 from the superior vena cava, and portions of the device which corresponding to implanted positions (e.g., superior vena cava (SVC), right atrium (RA), inferior vena cava (IVC)). A skirt 136 is also shown (which can include a corresponding reinforcing structure 125, which may be a stent structure, which can be interconnected with or integral with the stent structure of the device or tubular body thereof itself. The skirt 136 can be arranged at a connection point between the right atrium and the inferior vena cava and be shaped such that pressure generated within the right atrium pushes flanges of the skirt 136 against the cardiac wall, thus sealing the TTD 100 and further minimizing backflow of regurgitated blood in the venous system.

In some embodiments, and as described previously, the outflow port is configured to receive a valve to control blood flow from the inferior vena cava and the superior vena cava into the right atrium and/or the right ventricle (e.g., in some embodiments, similar to that which the native tricuspid valve). The valve within the outflow port can be a prosthetic valve or an allograft or a xenograft transplant (e.g., human, bovine, porcine). In some embodiments, the outflow port can be configured such that valves implanted therein are replaceable (in some embodiments, by removal of a previously placed valve, and in some embodiments, affixing or otherwise placing a new valve within the previously placed valve).

In some embodiments, the TTD can be mounted into a transcatheter delivery system and deployed out of it.

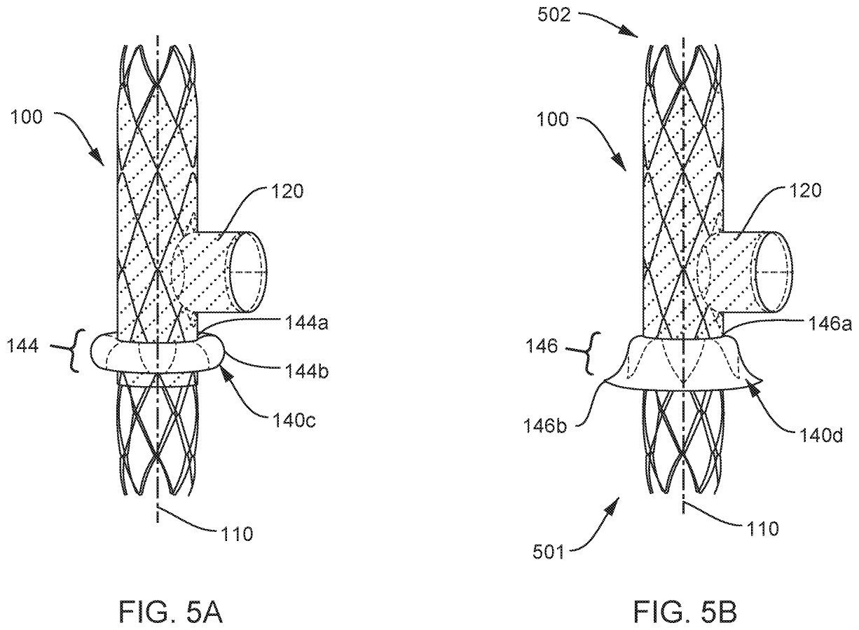

FIGS. 4A-B and FIGS. 5A-B illustrate various configurations of skirt sealing structures 140a through 140d, according to some embodiments. FIG. 4A illustrates a skirt 140a having a first flange shape with an inner diameter 141a which is attached to the tubular body proximate to the first end 401 and an outer diameter 141b, material connected therebetween interfacing with the wall of the right atrium. In embodiments, the outer diameter 141b may be spaced longitudinally (relative to the longitudinal axis 110) away from the inner diameter 141a in a direction toward the second end 402, the outer diameter 141b including a straight edge. The skirt structure, in some embodiments, is configured to prevent backflow from the right atrium into the inferior vena cava.

In some embodiments, the skirt structure is designed to prevent backflow from the right atrium into the inferior vena cava toward the first end 401 while allowing unblocked flow from the hepatic vein into the inferior vena cava, appreciating the proximity of the hepatic vein to the inferior vena cava. As seen in FIG. 1A, if a structure accomplishing the same function as the skirt structure employed at a position distal to the right atrium and along the inferior vena cava, it is possible that the hepatic vein would be occluded. To avoid this, the present disclosure presents the skirt structures of FIG. 4A through FIG. 5B. In embodiments, the skirt material can be made of any biocompatible woven or sheet fabric or polymer, and/or biological tissue.

FIG. 4B illustrates a skirt 140b that includes an outer diameter 142b, which is for interfacing with the inferior vena cava at the first end 401, and an inner diameter 142a spaced longitudinally (relative to the longitudinal axis 110) away from the outer diameter 142b in a direction toward the second end 402, the outer diameter 142b including a curved edge 142c.

FIG. 5A illustrates a skirt 140c that includes a donut shape 144 having an outer diameter 144b configured to interface with the inferior vena cava and an inner diameter 144a radially spaced (relative to the longitudinal axis 110) away from the outer diameter 144b and configured for attachment to the tubular body of the TTD 100.

FIG. 5B illustrates a skirt 140d that includes a flange shape 146, which includes an outer diameter 146b arranged proximate to the first end 501 and for interfacing with the inferior vena cava, and an inner diameter 146a which is attached to the tubular body and spaced longitudinally (relative to the longitudinal axis 110) away from the inner diameter 146a in a direction toward the second end 502, the outer diameter 146b including a straight edge.

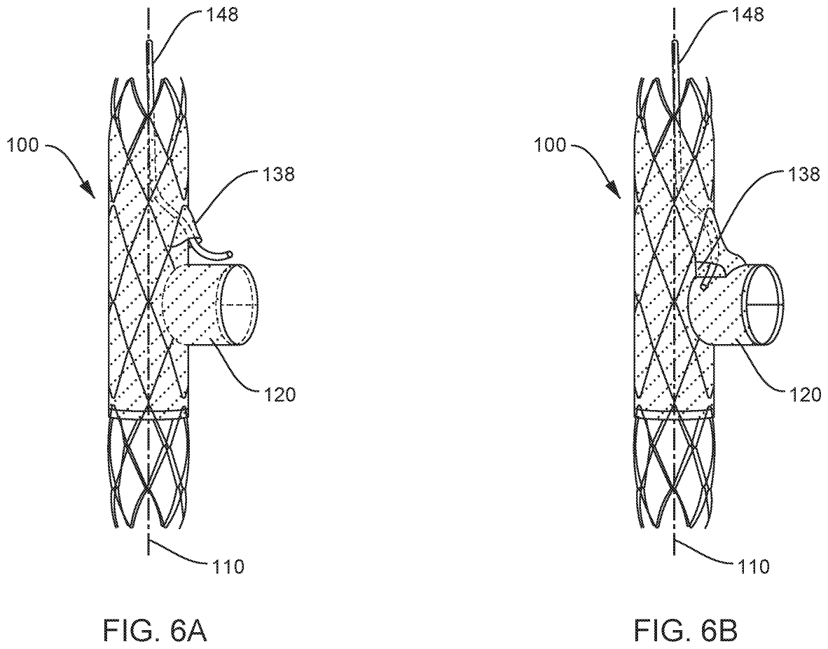

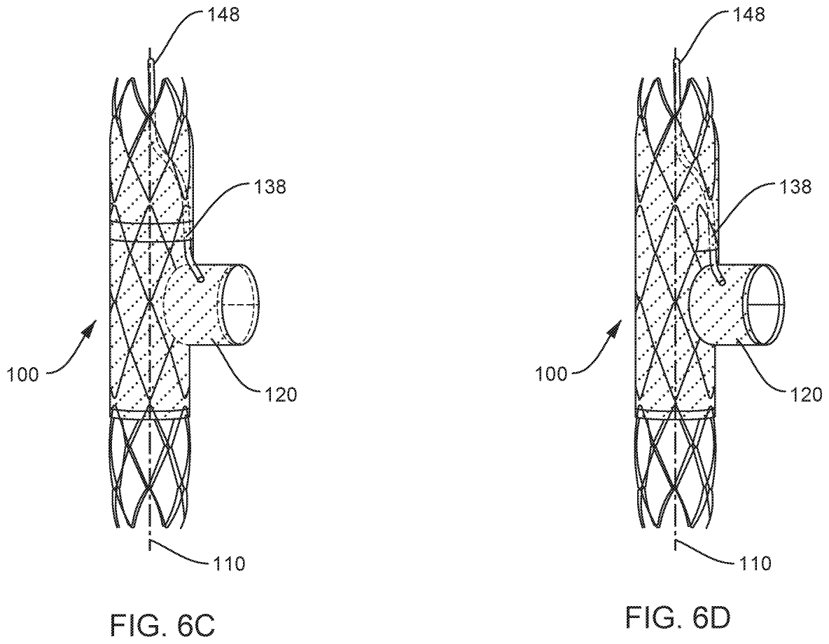

FIGS. 6A-D are side views of ports or valves 138 configured for placement of other devices, through a wall of the TTD 100, according to embodiments. In some embodiments, the port or valve 138 can be configured to receive leads 148 from a pacemaker, cardiac resynchronization therapy (CRT) device, or defibrillator device. Such leads 148, for example, can be inserted into such a port 138, without requiring such leads to pass through a working valve (which causes inefficiencies and potential damage).

According to some embodiments, the port/valve 138 can be configured to allow antegrade lead insertion into the right atrium and right ventricle while preventing retrograde flows.

This port/valve 138 can be referred to as a PM port. In some embodiments, the port/valve 138 can be an opening, an opening with a flap—which may simply be overlapping portions of a sidewall of the TTD 100, which may be curved (FIG. 6D), straight (FIG. 6C), a slot in the side of the TTD 100, a slot in the side of the outflow port 120 (FIG. 6B), and/or a covered opening (FIG. 6A).

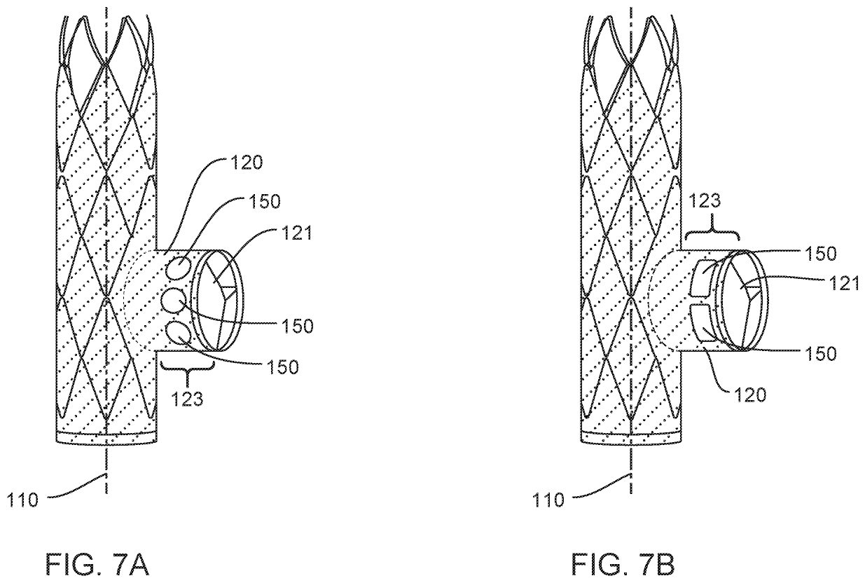

FIGS. 7A and 7B are side views of a TTD 100 and, particularly, the outflow port 120. In some embodiments, as shown, all or a portion of the sidewalls 123 of the outflow port 120 can include openings/fenestrations 150, which can be configured to allow backflow of blood to reach a valve 121 arranged within the outflow port (i.e., such a valve encounter blood flow more than just “head-on”/directly, but via the side. In other words, the openings/fenestrations 150 may be on the atrial side of the valve 121. Such a position permits the valve 121 to open and close with pressure changes in the heart and in view of impacts of tricuspid regurgitation.

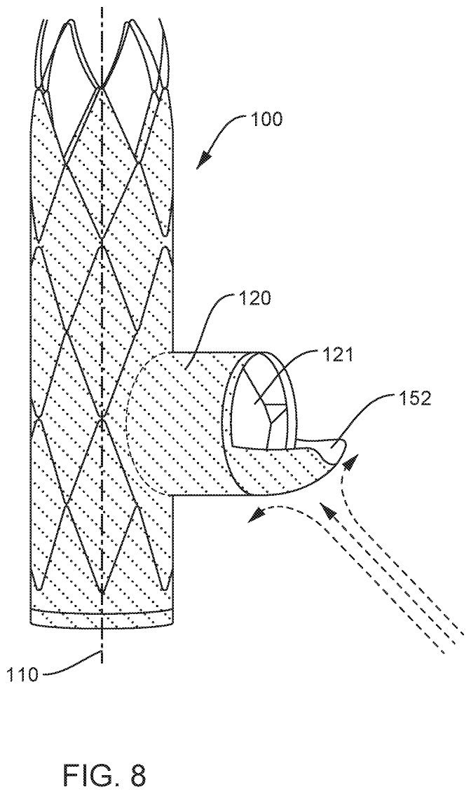

FIG. 8 is a cross-sectional side view of a TTD 100, and a blocking feature 152 arranged adjacent to an outflow port 120 thereof, according to some embodiments. In some embodiments, the blocking feature 152 is configured as an aid in blocking a flow of blood (in some embodiments, a jet of blood) from the native tricuspid valve 128. For instance, the blocking feature 152 redirects regurgitated blood that would otherwise flow directly into the outflow port 120 and would, in the event a valve 121 resides within the outflow port 120, possibly prevent the valve 121 from properly functioning. Such a blocking feature, or stopper device, can be, for example, a flap of flexible or rigid material placed on the distal end of the outflow port 120.

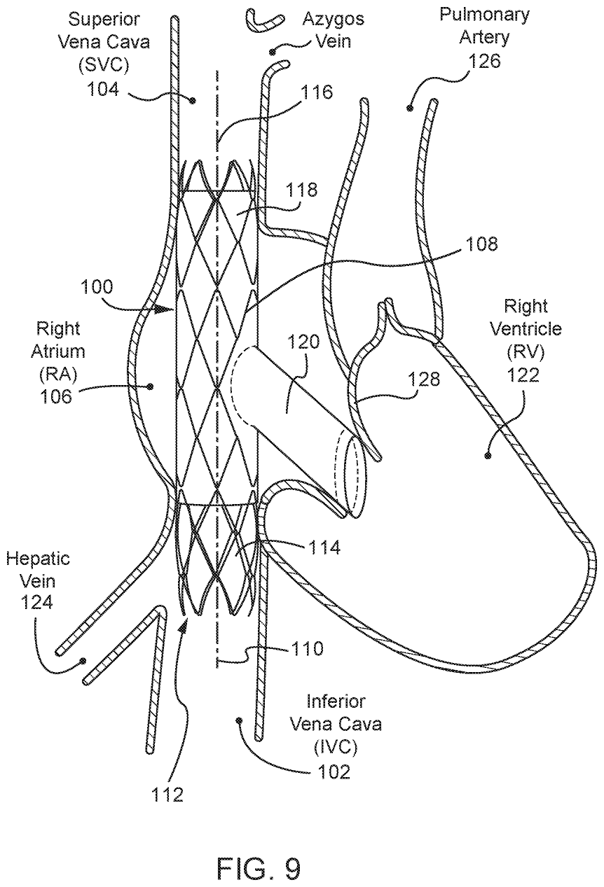

FIG. 9 is a side view of a TTD 100 implanted into the inferior vena cava 102, the superior vena cava 104, right atrium 106, and right ventricle 122, according to some embodiments. As shown, in some embodiments, the distal end of the outflow port 120 projects past the native tricuspid valve 128 and into the right ventricle 122.

FIGS. 10A-B illustrate side views of a TTD 100 showing different configurations of the outflow port 120 and components thereof, and in particular, the outflow port 120 being, in some embodiments, at an angle such that the distal end is projecting toward the apex of the heart (i.e., upward projecting), and, in some embodiments, the outflow port 12—being at an angle such that the distal end is projecting toward the base of the heart (i.e., downward projecting).

FIGS. 11A-D illustrate side views of a TTD 100, showing different configurations of the outflow port 120, and components thereof, following delivery and/or implantation of a valve 121 to the outflow port 120. Illustrations of delivery and/or implantation of the valve 121 to the outflow port 120 of the TTD 100 are shown in FIG. 11E to FIG. 11G and will be described below.

The TTD 100 is arranged within the superior vena cava, the inferior vena cava, and the right atrium immediately upon delivery, via catheter, to the heart. As shown in FIG. 11E, upon delivery, the TTD 100 may be deployed, or expanded, to fill the luminal space of the superior vena cava and the inferior vena cava and to project into the right atrium via the outflow port (OP). Once the TTD 100 is expanded, a valve (V) may be delivered to the TTD 100, as shown in FIG. 11F, wherein the V is directed toward the OP. Once in position, the V may be deployed to occupy the luminal space of the OP, as shown in FIG. 11G. Deploying the V may include self-expansion of the V, balloon expansion of the V, and the like. In FIG. 11G, the V is expanded by balloon expansion via inflation of a balloon (B).

In view of the above, embodiments of the present disclosure include a method of implanting a valve within a tubular stent, the method comprising delivering the TTD 100 to the heart of a patient, such that upon implantation, a first end and associated first end portion of the tubular body is arranged in the inferior vena cava, a second end and associated second end portion of the tubular body is arranged in the superior vena cava, and the first outflow port OP is arranged at least in the right atrium, delivering a stented valve within the TTD 100 and up through one or the other of the inferior vena cava and the superior vena cava so as to be arranged within at least a portion of the first outflow port OP, and balloon expanding the stented valve V within the first outflow port OP. In an embodiment, the expanding the stented valve V within the first outflow port OP includes greater expansion on at least one first portion of the stented valve V than remaining portions of the stented valve V. In embodiments, the first portion comprises at least one of and optionally two of a proximal portion, a center portion, and a distal portion of the stented valve V.

It should be appreciated that while a valve can be provided as an integral component with the TTD 100 or implanted at the time the TTD 100 is deployed, the TTD 100 can also be deployed without a valve and can be later modified, if desired. In embodiments, during or after a surgery to deploy the TTD 100, it may be determined that a valve is needed within the outflow port. To this end, during or after the surgery to deploy the TTD 100, any type of valve may then be delivered to the outflow port and implanted therein. The valve in FIG. 11E through FIG. 11G is an example of any off the shelf valve that is used for cardiac application (e.g., a SAPIEN 3 transcatheter heart valve), and is merely used for illustration of how a valve can be delivered within the TTD 110.

In view of the above, and with reference now to FIG. 11A, the outflow port 120 can be configured with at least one restriction, configured as a bottle-neck structure 156, according to some embodiments. The bottleneck structure 156 can be between the proximal end and the distal end of the outflow port 120, and, in some embodiments, can be configured as an hour-glass shape (i.e., the walls of the outflow port), with the bottleneck structure 156 being within the hourglass structure. As such, the bottleneck structure 156 can be configured to prevent the migration of a valve 121 arranged within the outflow port 120. To this end, during deployment of the valve 121, as in FIG. 11G, a middle region of the valve 121 may be cinched by the bottleneck structure 156 to prevent translation of the valve 121 within the outflow port 120.

In some embodiments, and with reference now to FIG. 11B, the outflow port 120 can include at least one of a first proximal restriction (at the base of the outflow port 120, and not shown) and a second distal restriction 155, each configured to prevent migration of a valve 121 arranged within the outflow port 120 (in some embodiments, the outflow port can include a single proximal or distal restriction).

In an embodiment, each restriction, as described with respect to FIG. 11B, can be configured as a terminus of the sidewall of the outflow port 120, as shown in FIG. 11D. To this end, the TTD 100 of FIG. 11D is configured such that the outflow port 120 includes a ballooning center 158c portion having a diameter that is larger than at least one of the diameters of the proximal end 158a and the distal end 158b thereof, the diameters of the proximal end 158a and the distal end 158b being configured to relative to a diameter of a valve 121 in order to prevent translation of the valve 121.

FIG. 11C illustrates a blocking structure (which may be referred to as a limiting structure), according to some embodiments, which may be configured as a cross-member 160, spanning across the diameter (or a portion thereof) of the distal end of the outflow port 120 (in some embodiments, such a cross-member may also be arranged on a proximal end of the outflow port 120, in place of or in addition to the cross-member placed on the distal end). The cross-member may be a wire structure (which may be connected with the stent structure of the tubular body 108, and/or stent structure associated with the outflow port 120), or other material that is affixed to the outflow port 120 (distal and/or proximal ends thereof). As with the blocking structure of FIG. 8, this feature is meant to modify blood flow and pressure gradients applied directly to the valve 121 within the outflow port 120 and which may impact function of the valve 121.

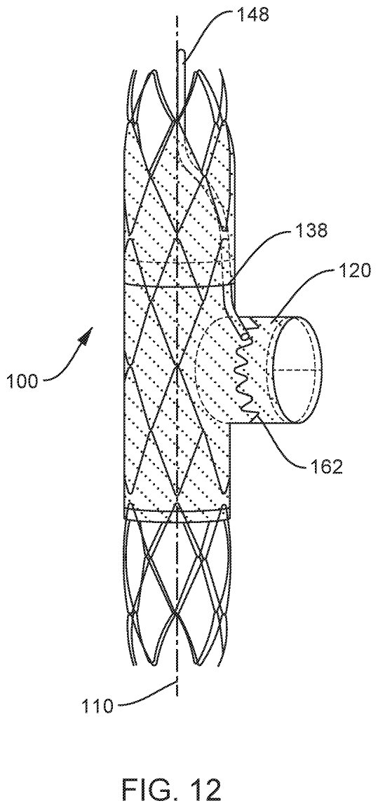

FIG. 12 is a side view of a TTD 100 illustrating a component contained among the outflow port 120 thereof. Specifically, in some embodiments, the walls of the outflow port may include a spring structure 162 (which may be configured as a stent like structure, and/or may be configured as part of a stent structure of the tubular body and/or outflow port). Such a spring structure 162, according to some embodiments, is configured to provide an inward radial force on the outflow port 120, which can be used to aid in, for example, retaining a valve within the outflow port 120.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all structure, parameters, dimensions, materials, functionality, and configurations described herein are meant to be an example and that the actual structure, parameters, dimensions, materials, functionality, and configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the claims supported by the present disclosure, and equivalents thereto, inventive embodiments may be practiced otherwise than as specifically described and claimed. Inventive embodiments of the present disclosure are also directed to each individual feature, system, article, structure, material, kit, functionality, step, and method described herein. In addition, any combination of two or more such features, systems, articles, structure, materials, kits, functionalities, steps, and methods, if such are not mutually inconsistent, is included within the inventive scope of the present disclosure. Some embodiments may be distinguishable from the prior art for specifically lacking one or more features/elements/functionality (i.e., claims directed to such embodiments may include negative limitations).

Also, as noted, various inventive concepts are embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to) for a particular embodiment(s).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined.

Other elements may optionally be present other than the elements specifically identified by the “and/or” clause, whether related or unrelated to those elements specifically identified. Thus, as a non-limiting example, a reference to “A and/or B”, when used in conjunction with open ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

“Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### NUMBERED EMBODIMENTS OF THE INVENTION

Notwithstanding the appended claims, the disclosure sets forth the following numbered embodiments:
(1) A stent graft device configured for implantation into the inferior vena cava and the superior vena cava of a patient to treat a diseased or otherwise damaged tricuspid valve of the patient, the device comprising a tubular body having a longitudinal axis and configured for implantation within the superior vena cava (SVC) and the inferior vena cava (IVC), such that, upon implantation, a first end and associated first end portion of the tubular body is arranged in the IVC, and a second end and associated second end portion of the tubular body is arranged in the SVC, a first outflow port configured for arrangement after implantation in the right atrium and right ventricle, and configured for the fixation of a surgical or transcatheter valve, wherein the first outflow port is arranged laterally to the longitudinal axis, and optionally, automatically deploys upon exiting a delivery catheter.
(2) The device of (1), wherein the first outflow port is arranged laterally to the longitudinal axis and at an oblique angle to the longitudinal axis.
(3) The device of either one of (1) or (2), wherein the first outflow port is arranged lateral to the longitudinal axis and at an oblique angle to the longitudinal axis, with a distal end of the first outflow port pointing in a direction toward the base of the heart.
(4) The device of any one of (1) to (3), wherein the first outflow port is arranged lateral to the longitudinal axis and at an oblique angle to the longitudinal axis, with a distal end of the first outflow port pointing in a direction toward the apex of the heart.
(5) The device of any one of (1) to (4), further comprising a material configured to at least partially cover the tubular body.
(6) The device of any one of (1) to (5), wherein the material is arranged on the tubular body such that it prevents blood flow through at least side portions of the tubular body.
(7) The device of any one of (1) to (6), wherein arrangement of the material on the tubular body is configured to allow inflow of blood from the azygos/innominate and/or hepatic veins.
(8) The device of any one of (1) to (7), further comprising a pacemaker/ICD lead port and/or valve (PLP) configured to receive for pacemaker/ICD lead passage.
(9) The device of any one of (1) to (8), wherein the PLP is arranged on a side of the tubular body.
(10) The device of any one of (1) to (9), wherein the first outflow port includes at least one of a first restriction and a second restriction, each configured to prevent migration of a valve arranged therein.
(11) The device of any one of (1) to (10), wherein the first outflow port includes at least one restriction, configured as a bottleneck structure, the bottleneck structure being within the first outflow port, or, the first outflow port is configured with an hourglass shape such that the bottleneck structure is within the hourglass structure, the bottleneck structure configured to prevent migration of a valve arranged within the first outflow port.
(12) The device of any one of (1) to (11), wherein the first outflow port includes a first stopper position, arranged on a distal end of the first outflow port, the first stopper portion configured to prevent migration of a valve arranged within the first outflow port away from the tubular body.
(13) The device of any one of (1) to (12), wherein the first outflow port includes a first stopper portion, arranged on a proximal end of the first outflow port, the first stopper portion configured to prevent migration of a valve arranged within the first outflow port toward the tubular body.
(14) The device of any one of (1) to (13), wherein the first outflow port includes a first stopper position arranged on a distal end of the first outflow port, the first stopper portion configured to prevent migration of a valve arranged within the first outflow port away from the tubular body, and a second stopper portion, arranged on a proximal end of the first outflow port, the second stopper portion configured to prevent migration of a valve arranged within the first outflow port toward the tubular body.
(15) The device of any one of (1) to (14), wherein the first outflow port includes a limiting structure arranged on a distal end of the first outflow port, the limiting structure configured to prevent migration of a valve arranged within the first outflow port away from the tubular body.
(16) The device of any one of (1) to (15), wherein at least one of a proximal end and a distal end of the first outflow port includes a diameter less than a diameter of a central portion of the first outflow port, which are configured to prevent migration of a valve arranged within the first outflow port.
(17) The device of any one of (1) to (16), wherein the first outflow port includes a stent structure configured as a spring and configured to provide an inward radial force on the first outflow port.
(18) The device of any one of (17), further comprising a skirt comprised of sealing material and arranged at least one of in and around the tubular body.
(19) The device of any one of (1) to (18), wherein the skirt is configured to prevent a backflow of blood from the right atrium to the inferior vena cava.
(20) The device of any one of (1) to (19), wherein the skirt includes a flange shape having an inner diameter which is attached to the tubular body proximate to the first end, and outer diameter for interfacing with the inferior vena cava, the other diameter spaced longitudinally away from the inner diameter in a direction toward the second end, outer diameter including a straight edge.
(21) The device of any one of (1) to (20), wherein the skirt includes an outer diameter which is for interfacing with the inferior vena cava, and an inner diameter spaced longitudinally away from the outer diameter in a direction toward the second end, the outer diameter includes a curved edge.
(22) The device of any one of (1) to (21), wherein the skirt includes a donut shape including an outer diameter configured to interface with the inferior vena cava, and an inner diameter radially spaced away from the outer diameter and configured for attachment to the tubular body.
(23) The device of any one of (1) to (22), wherein the skirt includes a flange shape, which includes an outer diameter arranged proximate the first end and for interfacing with the inferior vena cava, and an inner diameter which is attached to the tubular body and spaced longitudinally away from the inner diameter in a direction toward the second end, the outer diameter including a straight edge.
(24) The device of any one of (1) to (23), wherein the first port is self-expanding and/or includes a predetermined shape.
(25) The device of any one of (1) to (24), wherein the device is configured for delivery to the IVC/SVC via a sheathing delivery device, and wherein, the device is optionally configured for re-sheathing by the sheathing delivery device for at least one of repositioning, redeploying, and removal.
(26) The device of any one of (1) to (25), wherein at least the first outflow port is configured to be re-sheathed in a sheathing device.
(27) The device of any one of (1) to (26), wherein the first outflow port includes a stent structure configured to support a valve device placed within, on, or adjacent to the first outflow port.
(28) The device of any one of (1) to (27), wherein the first outflow port is configured with a length and/or shape such that upon implantation, the first port reaches the right atrium.
(29) The device of any one of (1) to (28), wherein the first outflow port includes at least one fenestration along a first contour (RA contour) or by having a shape that allows flow from the sides of a valve within, on, or adjacent the first port.
(30) The device of any one of (1) to (29), wherein the first outflow port is designed to prevent retrograde jet flows from reaching a/the valve placed within, on, or adjacent to the first outflow port.
(31) The device of any one of (1) to (30), wherein, upon implantation of the device, at least a portion of the first port is arranged within the RA, and optionally, at least a portion of the first outflow port protrudes into the right ventricle via the tricuspid valve.
(32) The device of any one of (1) to (31), wherein the tubular body includes a stent comprising a scaffold structure configured to operate in a first compressed mode, such that the device fits within a delivery catheter, and a second expanded mode, such that, upon placement of the device at an implantation site, the device is configured to self-expand therein, or expand via a balloon.
(33) The device of any one of (1) to (32), wherein the tubular body includes a rigidity that varies at different portions thereof.
(34) A method of delivering the device of any one of (1) to (33) via at least one of a transcatheter delivery device and a transapical delivery device.
(35) A method of implanting a valve within a tubular stent, the method comprising delivering a stent graft device according to any of claims 1-33 to the heart of a patient, such that upon implantation, a first end and associated first end portion of the tubular body is arranged in the IVC, a second end and associated second end portion of the tubular body is arranged in the SVC, and the first outflow port is arranged at least in the right atrium, delivering a stented valve up through one or the other of the IVC and SVC so as to be arranged within at least a portion of the first outflow port, and balloon expanding the stented valve within the first outflow port.
(36) The method of (35), wherein balloon expanding the stented valve within the first outflow port includes greater expansion on at least one first portion of the stented valve than remaining portions of the valve.
(37) The method of either one of (35) or (36), wherein the first portion comprises at least one of and optionally two of a proximal portion, a center portion, and a distal portion of the stented valve.
(38) A system, device, apparatus, or method according to any of the disclosed embodiments.

1. A stent graft device configured for implantation into the inferior vena cava and the superior vena cava of a patient to treat a diseased or otherwise damaged tricuspid valve of the patient, the device comprising:
a tubular body having a longitudinal axis and configured for implantation within the superior vena cava (SVC) and the inferior vena cava (IVC), such that, upon implantation, a first end and associated first end portion of the tubular body is arranged in the IVC, and a second end and associated second end portion of the tubular body is arranged in the SVC;
a first outflow port configured for arrangement after implantation in the right atrium and right ventricle, and configured for the fixation of a surgical or transcatheter valve, wherein the first outflow port:
is arranged laterally to the longitudinal axis; and
optionally, automatically deploys upon exiting a delivery catheter.

2. The device of claim 1, wherein the first outflow port is arranged laterally to the longitudinal axis and at an oblique angle to the longitudinal axis.

3. The device of claim 1 or 2, wherein the first outflow port is arranged lateral to the longitudinal axis and at an oblique angle to the longitudinal axis, with a distal end of the first outflow port pointing in a direction toward the base of the heart.

4. The device of claim 1 or 2, wherein the first outflow port is arranged lateral to the longitudinal axis and at an oblique angle to the longitudinal axis, with a distal end of the first outflow port pointing in a direction toward the apex of the heart.

5. The device of any of claims 1-4, further comprising a material configured to at least partially cover the tubular body.

6. The device of claim 5, wherein the material is arranged on the tubular body such that it prevents blood flow through at least side portions of the tubular body.

7. The device of claim 5 or 6, wherein arrangement of the material on the tubular body is configured to allow inflow of blood from the azygos/innominate and/or hepatic veins.

8. The device of any of claims 1-7, further comprising a pacemaker/ICD lead port and/or valve (PLP) configured to receive for pacemaker/ICD lead passage.

9. The device of claim 8, wherein the PLP is arranged on a side of the tubular body.

10. The device of any of claims 1-9, wherein the first outflow port includes at least one of a first restriction and a second restriction, each configured to prevent migration of a valve arranged therein.

11. The device of any of claims 1-9, wherein the first outflow port includes at least one restriction, configured as a bottleneck structure, the bottleneck structure being within the first outflow port, or, the first outflow port is configured with an hourglass shape such that the bottleneck structure is within the hourglass structure, the bottleneck structure configured to prevent migration of a valve arranged within the first outflow port.

12. The device of any of claims 1-9, wherein the first outflow port includes a first stopper position, arranged on a distal end of the first outflow port, the first stopper portion configured to prevent migration of a valve arranged within the first outflow port away from the tubular body.

13. The device of any of claims 1-9, wherein the first outflow port includes a first stopper portion, arranged on a proximal end of the first outflow port, the first stopper portion configured to prevent migration of a valve arranged within the first outflow port toward the tubular body.

14. The device of any one of claims 1-9, wherein the first outflow port includes a first stopper position arranged on a distal end of the first outflow port, the first stopper portion configured to prevent migration of a valve arranged within the first outflow port away from the tubular body, and a second stopper portion, arranged on a proximal end of the first outflow port, the second stopper portion configured to prevent migration of a valve arranged within the first outflow port toward the tubular body.

15. The device of any of claims 1-9, wherein the first outflow port includes a limiting structure arranged on a distal end of the first outflow port, the limiting structure configured to prevent migration of a valve arranged within the first outflow port away from the tubular body.

16. The device of any of claims 1-9, wherein at least one of a proximal end and a distal end of the first outflow port includes a diameter less than a diameter of a central portion of the first outflow port, which are configured to prevent migration of a valve arranged within the first outflow port.

17. The device of any of claims 1-9, wherein the first outflow port includes a stent structure configured as a spring and configured to provide an inward radial force on the first outflow port.

18. The device of any of claims 1-17, further comprising a skirt comprised of sealing material and arranged at least one of in and around the tubular body.

19. The device of claim 18, wherein the skirt is configured to prevent a backflow of blood from the right atrium to the inferior vena cava.

20. The device of claim 18 or 19, wherein the skirt includes a flange shape having an inner diameter which is attached to the tubular body proximate to the first end, and outer diameter for interfacing with the inferior vena cava, the other diameter spaced longitudinally away from the inner diameter in a direction toward the second end, outer diameter including a straight edge.

21. The device of any of claims 18-19, wherein the skirt includes an outer diameter which is for interfacing with the inferior vena cava, and an inner diameter spaced longitudinally away from the outer diameter in a direction toward the second end, the outer diameter includes a curved edge.

22. The device of any of claims 18-19, wherein the skirt includes a donut shape including an outer diameter configured to interface with the inferior vena cava, and an inner diameter radially spaced away from the outer diameter and configured for attachment to the tubular body.

23. The device of any of claims 18-19, wherein the skirt includes a flange shape, which includes an outer diameter arranged proximate the first end and for interfacing with the inferior vena cava, and an inner diameter which is attached to the tubular body and spaced longitudinally away from the inner diameter in a direction toward the second end, the outer diameter including a straight edge.

24. The device of any of claims 1-23, wherein the first port is self-expanding and/or includes a predetermined shape.

25. The device of any of claims 1-24, wherein the device is configured for delivery to the IVC/SVC via a sheathing delivery device, and wherein, the device is optionally configured for re-sheathing by the sheathing delivery device for at least one of repositioning, redeploying, and removal.

26. The device of any of claims 1-25, wherein at least the first outflow port is configured to be re-sheathed in a sheathing device.

27. The device of any of claims 1-26, wherein the first outflow port includes a stent structure configured to support a valve device placed within, on, or adjacent to the first outflow port.

28. The device of any of claims 1-27, wherein the first outflow port is configured with a length and/or shape such that upon implantation, the first port reaches the right atrium.

29. The device of any of claims 1-28, wherein the first outflow port includes at least one fenestration along a first contour (RA contour) or by having a shape that allows flow from the sides of a valve within, on, or adjacent the first port.

30. The device of any of claims 1-29, wherein the first outflow port is designed to prevent retrograde jet flows from reaching a/the valve placed within, on, or adjacent to the first outflow port.

31. The device of any of claims 1-30, wherein, upon implantation of the device, at least a portion of the first port is arranged within the RA, and optionally, at least a portion of the first outflow port protrudes into the right ventricle via the tricuspid valve.

32. The device of any one of claims 1-31, wherein the tubular body includes a stent comprising a scaffold structure configured to operate in a first compressed mode, such that the device fits within a delivery catheter, and a second expanded mode, such that, upon placement of the device at an implantation site, the device is configured to self-expand therein, or expand via a balloon.

33. The device of any of claims 1-32, wherein the tubular body includes a rigidity that varies at different portions thereof.

34. A method of delivering the device of any of claims 1-33 via at least one of a transcatheter delivery device and a transapical delivery device.

35. A method of implanting a valve within a tubular stent, the method comprising:
delivering a stent graft device according to any of claims 1-33 to the heart of a patient, such that upon implantation, a first end and associated first end portion of the tubular body is arranged in the IVC, a second end and associated second end portion of the tubular body is arranged in the SVC, and the first outflow port is arranged at least in the right atrium;
delivering a stented valve up through one or the other of the IVC and SVC so as to be arranged within at least a portion of the first outflow port; and
balloon expanding the stented valve within the first outflow port.

36. The method of claim 35, wherein balloon expanding the stented valve within the first outflow port includes greater expansion on at least one first portion of the stented valve than remaining portions of the valve.

37. The method of claim 36, wherein the first portion comprises at least one of and optionally two of a proximal portion, a center portion, and a distal portion of the stented valve.

38. A system, device, apparatus, or method according to any of the disclosed embodiments.
